(19) United States
(12) Patent Application Publication
(10) Pub. No.: US 2013/0123694 A1
(43) Pub. Date: May 16, 2013

(54) DIAGNOSTIC KIT AND METHOD FOR MEASURING BALLOON DIMENSION IN VIVO

(71) Applicant: Angiometrix Corporation, Bethesda, MD (US)

(72) Inventors: Raghavan SUBRAMANIYAN, Bangalore (IN); Goutam DUTTA, Bangalore (IN); Venugopal GOPINATHAN, Bangalore (IN)

(73) Assignee: ANGIOMETRIX CORPORATION, Bethesda, MD (US)

(21) Appl. No.: 13/709,311

(22) Filed: Dec. 10, 2012

Related U.S. Application Data
(63) Continuation of application No. PCT/US2011/040158, filed on Jun. 13, 2011.
(60) Provisional application No. 61/383,744, filed on Sep. 17, 2010.

(51) Int. Cl. A61M 25/10

(52) U.S. Cl. 604/100.01

(57) ABSTRACT

A method for measuring a balloon expansion profile in vivo is provided. The method comprises providing a balloon with at least one sensing element as a diagnostic device, where the at least one sensing element is characterized by at least one attribute that is representative of balloon dimension; measuring the at least one attribute to obtain an observed attribute value; and estimating the balloon dimension and the balloon expansion profile based on the observed attribute value. A diagnostic kit for measuring a balloon expansion profile in vivo is also provided. The diagnostic kit comprises the diagnostic device; a measurement module for measuring an observed attribute value for the attribute; and a processor module for processing the observed attribute value to estimate the balloon expansion profile as one or more outputs.

### CROSS-REFERENCE TO RELATED APPLICATIONS

This application is a continuation of International Patent Application No. PCT/US2011/040158 filed Jun. 13, 2011 which claims the benefit of U.S. Provisional Patent Application No. 61/383,744 tiled Sep. 17, 2010 to Gopinathan, and also claims the benefit of foreign priority of Indian Provisional Patent Application No. 1636/CHE/2010, filed Jun. 13, 2010 to Gopinathan et al., both entitled “Systems and Methods for Measurements of Lumen Parameters”, the disclosures of which are incorporated by reference herein.

### TECHNICAL HELD

The invention relates generally to the field of medical diagnostic and more specifically to balloon catheters.

### BACKGROUND

Catheter as used in medical diagnostics refers to a tube that can be inserted into a body cavity, duct, or vessel (referred herein generally as body lumen). Catheters are used in several clinical, procedures and allow drainage, administration of fluids or gases, or access by surgical instruments in different body lumens. The process of inserting a catheter in the desired body lumen is called catheterization.

A specific category of catheter called balloon catheter have an inflatable “balloon” at its tip which is used during a catheterization procedure to enlarge a narrow opening or passage within the body. During a medical procedure, the deflated balloon catheter is positioned in the body lumen, the balloon is inflated to perform the necessary procedure, and deflated again in order to be removed.

Balloon catheters are also utilized in the deployment of stents during angioplasty. For these procedures, the balloon catheters include a pre-mounted stent on the balloon. When the balloon is inflated the stent is also expanded. When the balloon is deflated the stent stays behind in the artery and the balloon catheter can be removed. Stems that are used in conjunction with a balloon catheter are known as balloon expandable stents.

During balloon angioplasty and stent deployment, a balloon is expanded by applying pressure to the fluid contained in the balloon through means provided outside the body of the subject undergoing the procedure. In both procedures, it is clinically important to know bow much the balloon has expanded. In angioplasty, the balloon expansion would directly be related to the expanded wall of the vessel around the balloon. In stent deployment the balloon expansion is directly related to the expanded size of the stent around it.

Each balloon comes with a nominal mapping of pressure versus balloon diameter based on the physical properties of the balloon. However, the actual expanded diameter of the balloon also depends upon the various factors such as plaque morphology (calcified versus non-calcified), plaque burden (amount of plaque) and hence resistance offered by the wall varies. The balloons are also made of semi compliant material and therefore the balloon may stretch longitudinally against increased pressure or expand more in regions of lower resistance and less in regions offering higher wall resistance. Hence this mapping is not a reliable measure of the expanded size of the balloon.

Currently there are a few techniques as described below that have evolved to obtain the balloon diameter after expansion but they are limited in there scope due to the reasons mentioned hereinabove.

WO 2010042653 provides a system, device and method for utilizing stretchable active integrated circuits with inflatable bodies. The invention allows for such operative features to come into direct contact with body structures, such as the inner wall of a lumen, and is useful for measurements and delivery of therapy.

CN 201223393 relates to a graduated length measurement balloon catheter, which comprises a multi-way joint, an outer tube and art inner tube. The graduated length measurement balloon catheter is characterized in that a plurality of metal rings are arranged on the outer tube in the balloon to form scales. The metal scales on the outer tube are clear and visible in X-rays and can measure the length of the pathologic change that is useful for making decisions on diagnosis and treatment and surgical operation.

WO 2008042347 provides techniques for the diagnosis and treatment of a narrowing lumen with a smart balloon catheter. The smart balloon catheter includes pressure and diameter sensing features along with a feedback system to control the dilation of the balloon. Ambient pressure of the lumen is detected with multiple pressure sensors located on the distal end of the catheter and displayed on a monitoring device. Ambient pressure results are used to position the distal end of the catheter within the narrowing lumen. A controlled gradual, or stepwise, dilation of the balloon occurs. The pressure sensors detect the ambient pressure of the lumen outside of the balloon, and the pressure within the balloon. Distance sensors measure the distance between the center of the catheter and the expanded balloon surface. The diameter of the balloon at different cross-sections is determined and displayed on the monitoring device. The volume of the balloon, and the waist of the narrowing lumen, are determined. The rate of the dilation continues as a function of input provided by pressure and distance sensors.

US 2008033316 provides a system, catheter and method for measuring the cross-sectional areas and pressure gradients in any hollow organ, such as, for example, blood vessels. One embodiment of such a system includes: an impedance catheter capable of being introduced into a targeted site; a solution delivery source; a constant current source; a balloon inflation control device; and a data acquisition and processing system that receives conductance and/or pressure gradient data from the catheter and calculates the cross-sectional area of the targeted site. In one embodiment, the catheter has an inflatable balloon along its longitudinal axis, thereby enabling the breakup of any materials causing stenosis at the targeted site and/or distention and delivery of an optional stent into the targeted site.

WO 2005070061 provides a system for measuring physiologic characteristics for treating abnormal mucosa in the esophagus comprises a sizing device having an inflatable balloon on a distal end of a catheter that is inflated with an expansion medium to expand the balloon to engage the wall of the esophagus so that the internal cross-section can be calculated or measured. The sizing device may also include an infusion source for delivering the expansion medium and means for measuring the amount and pressure of the expansion medium inside the catheter.

WO 0137897 provides a sizing catheter and method of measuring a preselected internal opening within a patient to provide a rapid and precise determination of first and second stretched diameters of the preselected internal opening. The sizing catheter and method may be utilized to determine an appropriate sized device to be positioned within the preselected opening.

U.S. Pat. No. 6,010,511 provides methods and apparatus for determining cross-sectional dimensions of body lumens, such as the diameter of a blood vessel. According to one exemplary method, the diameter of a blood vessel is measured by first inflating, a balloon catheter within the lumen until the balloon diameter matches the lumen diameter. Inflation may be at a very low pressure and be constrained by the lumen, or may alternatively be controlled by monitoring, the flow within the lumen. The balloon includes at least one measurement element which indicates the expanded balloon cross-sectional area, circumference, or diameter.

U.S. Pat. No. 5,397,308 provides an improved balloon catheter for angioplasty and the like for measuring the inflation of a balloon after insertion into the body. A pair of electrodes is mounted in spaced relation within the balloon interior wall such that as the internal area within the balloon is varied by inflation of the balloon with an electrically conductive fluid, the electrodes monitor the changing electrical resistance between the electrodes. The electrodes are connected through the catheter to an external electrical measurement circuit for measuring the change in electrical resistance of the conducting fluid and thus determining the amount of balloon inflation. The change in resistance would be due to the average change in the diameter of the balloon as well as the average longitudinal expansion of the balloon.

The above described methods are used by physicians to ascertain the diameter of the expanded balloon through a combination of techniques that involve the mapping the measurement information, knowledge and experience, and an eyeball estimate of the balloon diameter from an X-Ray image (angiogram).

However, there continues to be a need for further improvement in the methods and techniques related to measurement of balloon dimensions for accurate delivery of stents and other procedures, as the techniques available today are all directed to obtaining the balloon diameter measurement at only few (usually one) specific locations and therefore inherently suffer from estimation errors. There is evidence showing poor correlation with angiographic assessment of expansion and actual expansion as measured by systems such as IVUS (intravascular ultrasound) and OCT (optical coherence tomography), and therefore an improved technique for measuring balloon expansion and dimensions thereof is needed.

### BRIEF DESCRIPTION

In one aspect, the invention provides a method for measuring a balloon expansion profile in vivo. The method comprises providing a balloon with at least one sensing element, wherein the at least one sensing element is characterized by at least one attribute that is representative of balloon dimension; measuring the at least one attribute to obtain an observed attribute value; and estimating the balloon dimension and the balloon, expansion profile based on the observed attribute value.

In another aspect, the invention provides a diagnostic kit for measuring a balloon expansion profile in vivo. The diagnostic kit comprises a balloon with at least one sensing element, where the at least one sensing element is characterized by at least one attribute that is representative of balloon dimension; a measurement module for measuring an observed attribute value for the attribute; and a processor module for processing the observed attribute value to estimate the balloon expansion profile as one or more outputs.

In yet another aspect, the invention provides a diagnostic device comprising a balloon having at least one sensing element for measuring at least one balloon expansion profile.

### BRIEF DESCRIPTION OF THE DRAWINGS

These and other features, aspects, and advantages of the present invention will become better understood When the following detailed description is read with reference to the accompanying drawings in which like characters represent like parts throughout the drawings, wherein:

FIG. 1 is a diagrammatic representation of an exemplary diagnostic device embodied as a balloon catheter that can be placed in vivo in a body lumen for measuring a balloon expansion profile;

FIG. 2 is a diagrammatic representation of another exemplary embodiment of the diagnostic device with a resistive element as a sensing element;

FIG. 3 is a diagrammatic representation of another exemplary embodiment of the diagnostic device with a resistive element that is fully integrated into the material of the balloon;

FIG. 4 is a diagrammatic representation of another exemplary embodiment of the diagnostic device;

FIGS. 5-7 are diagrammatic representations of other non limiting exemplary embodiments of the diagnostic device;

FIG. 8 is a flowchart representation of the exemplary method steps for measuring a balloon expansion profile in vivo;

FIG. 9 is a graphical representation between an attribute and a balloon dimension used for measuring a balloon expansion profile in vivo; and

FIG. 10 is a diagrammatic representation of an exemplary embodiment of a diagnostic kit for measuring a balloon expansion profile in vivo.

### DETAILED DESCRIPTION

As used herein and in the claims, the singular forms “a,” “an,” and the include the plural reference unless the context dearly indicates otherwise.

As used herein, lumen means the inner space of any tubular structured component of a subject such as a human being, such as an artery or intestine. For example, the interior of a vessel, such as the inner space in an artery or vein through which blood flows is considered a lumen. Similarly, a lumen may also represent the inside space of a cellular component or structure, such as the endoplasmic reticulum.

As used herein, angioplasty is the technique of mechanically widening a narrowed or obstructed blood vessel to aid improved blood flow in the blood vessel. Angioplasty may also involve stent deployment in the body lumen. Stents are composed of fine wire materials such as platinum that can be inserted through a thin catheter and expanded into a predetermined shape once they are guided into place.

Aspects of this invention relate to both balloon catheters for widening a narrowed or obstructed blood vessel and to balloon expandable stents that are used to deploy a stent in the body lumen as a part of medical treatment. The procedures related to such uses of catheters are generally referred herein as medical procedures.

As explained herein above, in order to accurately diagnose a constriction in a body passage like a blood vessel, and simultaneously perform constriction and dilation of the balloon and/or position a stent in the body lumen, it is important to know how much the balloon has inflated. The more accurate is the measurements for balloon expansion, the better is the diagnosis and medical procedure.

The exemplary embodiments of the invention incorporate sensing element or elements in the material of the catheter balloon or the angioplastic and stent delivery balloon, which react in a measurable manner to the expansion of the balloon. For example, when the balloon expands, at least one attribute for the balloon is measured that changes due to expansion, and the balloon expansion profile is so inferred. The attribute being measured could be voltage difference, electrical resistance, or resonance frequency or any other attribute that can be measured and is representative of a balloon dimension.

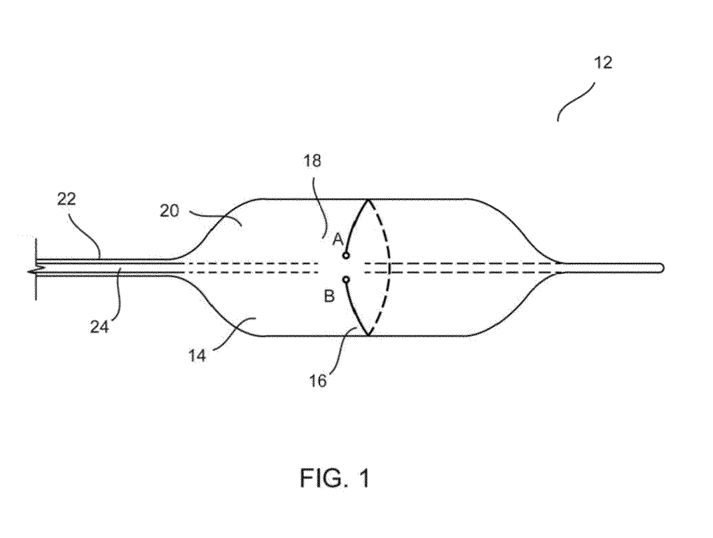

An exemplary embodiment of the invention is shown in FIG. 1 as a diagnostic device 12 comprising a balloon 14 having at least one sensing element 16 for measuring at least one balloon expansion profile. The sensing element 16 in the exemplary embodiment is in the form of an elastic resistive element embedded along at least a portion of a circumference on a surface of the balloon. The two end points of the elastic resistive element form terminals A and B and function as two spaced apart electrodes 18. The two spaced apart electrodes are the sub-elements that are used for making electrical measurements that are used to generate the balloon expansion profile, while the balloon is placed in-vivo via the catheter 22 and expanded by a pressurized fluid 20 through the conduit 24 during a medical procedure. The sensing element in the exemplary embodiment is shown as a ring like structure but other adaptations for placing the sensing element are possible and are included in the scope of the invention.

Some exemplary adaptations include, the sensing element being integral component of the balloon where the sensing element can be incorporated in the material used to construct the balloon, through known techniques. A range of polymers are used for the construction of catheters, including silicone rubber, latex, natural rubber latex and thermoplastic elastomers. In another more specific example the sensing element is a piezoelectric material integrated in the balloon, where change in electrical field is sensed by the piezoelectric material, in another specific example the sensing element is a capacitive element embedded in a wall of the balloon. The capacitive element may be incorporated by sandwiching a dielectric between two layers of balloon wall. Such a capacitive element would sense a change in capacitance when the diameter of the balloon changes. In another embodiment, instead of the resistive element, an inductive element such as a coil is used. In vet another embodiment, the balloon incorporates a material whose tension can be measured. The tension of the wall of the balloon is directly related to the diameter to which it has expanded. Such a tension could be measured indirectly by means such as sound vibrations as there is a natural frequency at which the taut balloon wall would vibrate.

In another exemplary embodiment a single sensing element is used whereas in yet another exemplary embodiment several sensing elements may be used. In the exemplary embodiment as shown in FIG. 1, the sensing element is incorporated as a discrete element (such as a ring or coil) that is embedded or attached to the inner or outer surface of the balloon. In a specific example multiple such rings can be along the longitudinal axis to obtain an expansion profile. The sensing element may be mounted on the surface of the balloon or may be present inside the balloon. In another embodiment a conductive ink is “painted” on the inner or outer surface of the balloon and is used as a sensing element. Radio opaque ink can also be used on the balloons that enable the method of placing such electrically active elements. Such an element can also be constructed by techniques that use conductive ink. Radio opaque ink can also be used to sense the balloon dimension.

In another embodiment similar to the ring sensing element, an element or multiple elements may be placed on the surface of the balloon parallel to the longitudinal axis to measure the longitudinal expansion of the balloon by measuring the diameter at different points along the axial length of the balloon to obtain the balloon expansion profile.

For example, in a conductance catheter two or more electrodes are placed along its length. When a high-frequency low-amplitude constant current is passed through the outer electrodes to generate an electric field, the potential difference between any pair of inner electrodes is used to calculate the balloon dimension and to generate the balloon expansion profile.

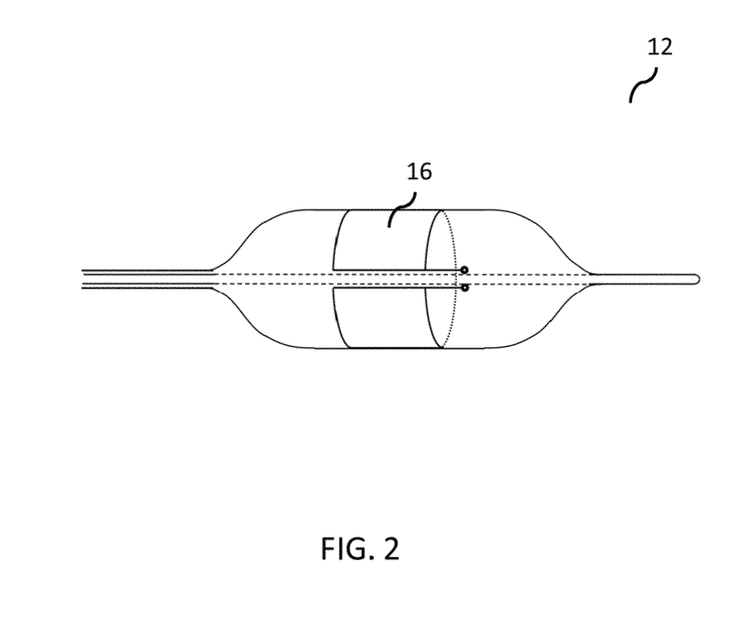

FIG. 2 is a diagrammatic representation of another exemplary embodiment of the diagnostic device 12 with a resistive element as a sensing element 16 comprising of a conductive material that could be a part of the construction material of the balloon. Multiple such elements could be used along the longitudinal axis.

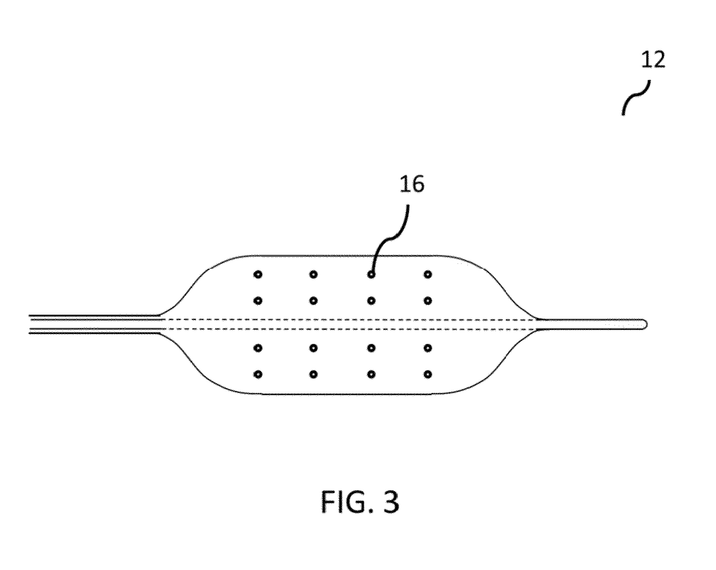

FIG. 3 is a diagrammatic representation of another exemplary embodiment of the diagnostic device 12 with a. resistive element 16 that is fully integrated into the material of the balloon. It would be appreciated by those skilled in the art that in such a configuration, the entire balloon surface is conductive, and the terminal electrodes are attached to the material of the balloon at various points. In this embodiment, the resistance could be measured between any two electrodes at a time.

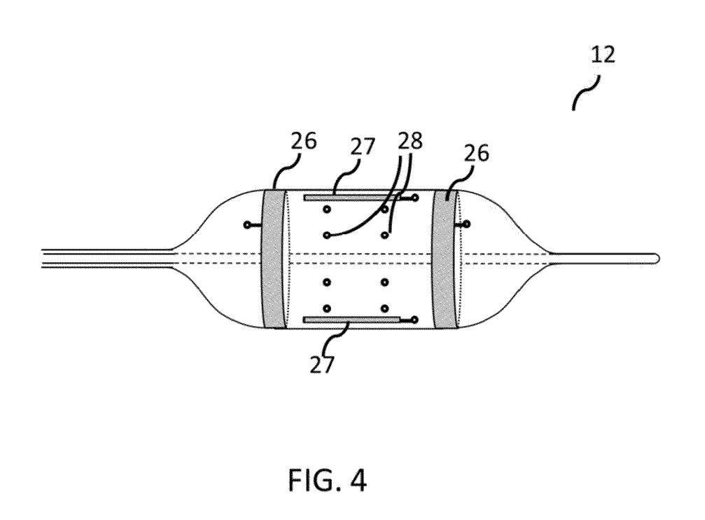

FIG. 4 is a diagrammatic representation of another exemplary embodiment 12 where a current is injected between a pair of electrodes 26 and or 27 at a time, and the voltage developed at multiple electrodes 28 is measured. As shown, the first pair of electrodes indicated by referral numeral 26 are the ring electrodes and the second pair of electrodes indicated by referral numeral 27 are strip electrodes that are laid out parallel to the axis of the balloon. In an exemplary implementation one of the pairs is excited at a time (26 or 27). By using the two pairs, the expansion of balloon in all directions can be determined. It would be appreciated by those skilled in the art that the measured voltage distribution can be used to map out the entire balloon expansion profile, both radially and longitudinally. As shown, the terminals used to inject current may be special electrodes that could be larger such as a highly conducting ring or a strip as shown in FIG. 4.

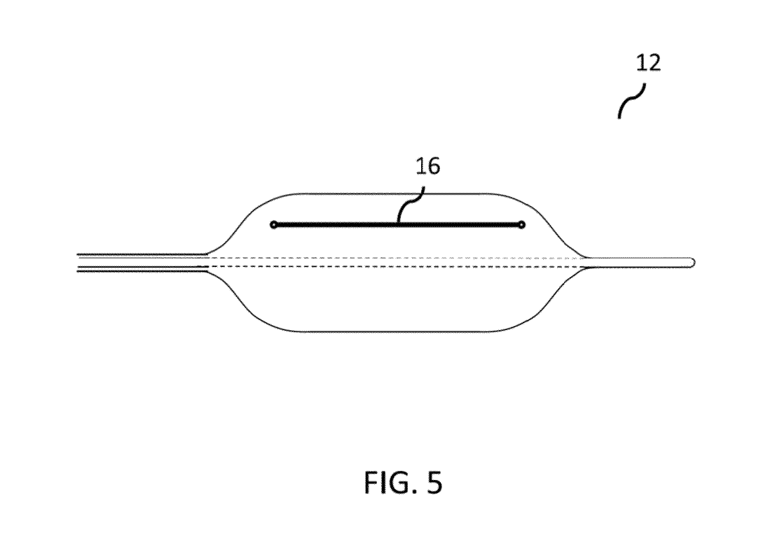

FIG. 5 is a diagrammatic representation of another exemplary embodiment 12 with a sensing element 16 placed longitudinally to measure expansion in length. Though only one sending element 16 is shown, more than one sensing elements may be used in specific implementations.

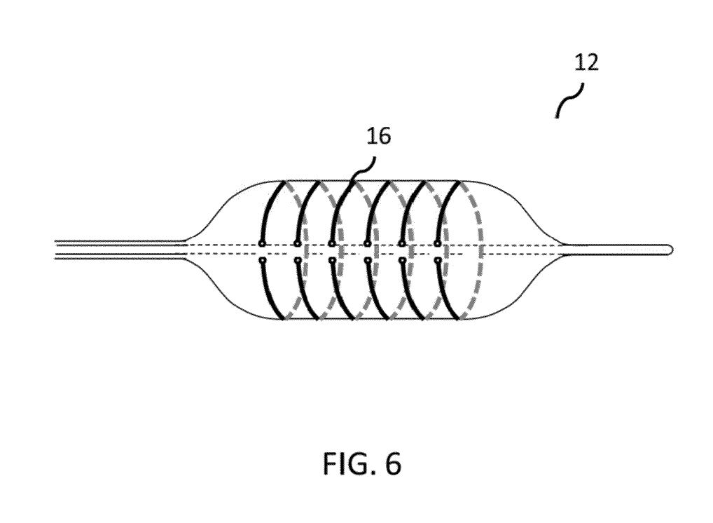

FIG. 6 is a diagrammatic representation of another exemplary embodiment 12 where multiple sensing elements 16 are placed spaced apart covering different cross-sectional areas of the balloon to obtain the balloon expansion profile taking into account that the different parts of the balloon may expand by different amounts, and the measurements from the multiple elements would yield the balloon expansion profile.

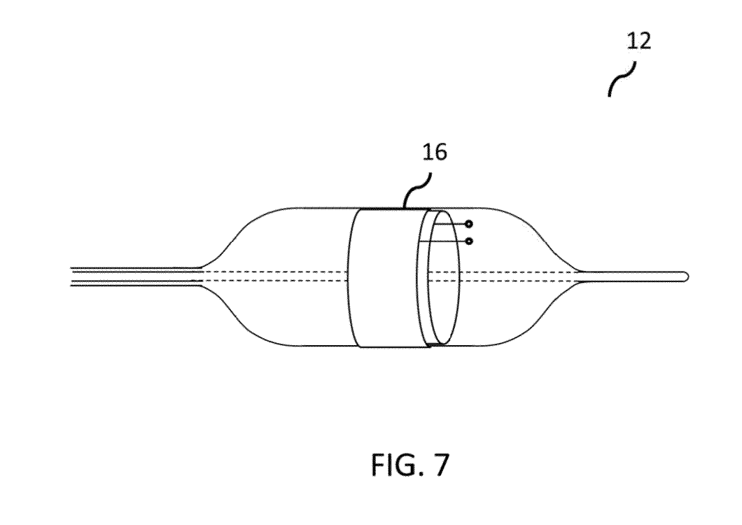

FIG. 7 is as diagrammatic representation of another exemplary embodiment 12 with the sensing element 16 which is in the form of a capacitance element comprising of two concentric cylindrical shells encapsulating a dielectric material. One terminal is connected to the outer layer of the concentric shell and one to the inner layer of the concentric shell as shown. More than one such concentric shells can be used placed inside the volume of the balloon.

It should be noted that the embodiments described herein are non-limiting examples and other adaptations may be implemented on the similar principles and are within the scope of the invention.

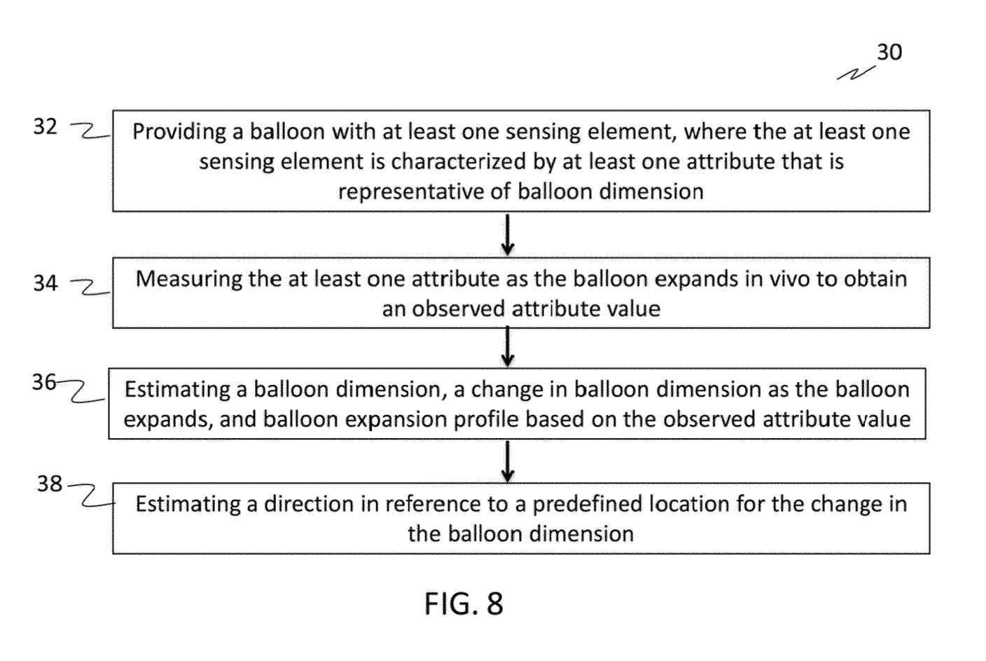

An aspect of the invention is the exemplary method for measuring a balloon expansion profile in vivo, the method being depicted generally by the flowchart 30 of FIG. 8. The method includes a step 32 of providing a balloon with at least one sensing element, where the at least one sensing element is characterized by at least one attribute that is representative of balloon dimension for example the embodiment of FIG. 1. The method further involves a step 34 for measuring the at least one attribute as the balloon expands in vivo to obtain an observed attribute value; and a step 36 for estimating a balloon dimension, a change in balloon dimension as the balloon expands, and balloon expansion profile based on the observed attribute value. The method includes measuring the observed attribute at a single location or at a plurality of locations.

It would be appreciated by those skilled in the art that the change in balloon dimension is representative of an expansion of the balloon. In one exemplary embodiment the method further includes a step 38 for estimating a direction in reference to a predefined location for the change in the balloon dimension. For example, the balloon expansion profile may be estimated as a dimension along one axis of the balloon, for example the longitudinal axis.

The attribute as referred herein could be electrical resistance or electrical impedance between at least two electrodes of FIG. 1. Impedance as used herein refers to the resistance (resistive impedance) of the element. However, as it would be appreciated by those skilled in the art the measurable property can also by inductance (inductive impedance) or capacitance (capacitive impedance) of the elastic resistive element, and are to be considered within the scope of the invention.

Now referring to the embodiment of FIG. 1, the two terminals A and B are used to measure the electrical impedance of the sensing element by drawing thin wires through the catheter. As the balloon expands, the sensing element expands with it. Thus the circumferential length of the sensing element increases. Simultaneously, there is a reduction in the cross sectional area of the sensing, element (the total volume being constant). Both these changes lead to an increase in impedance.

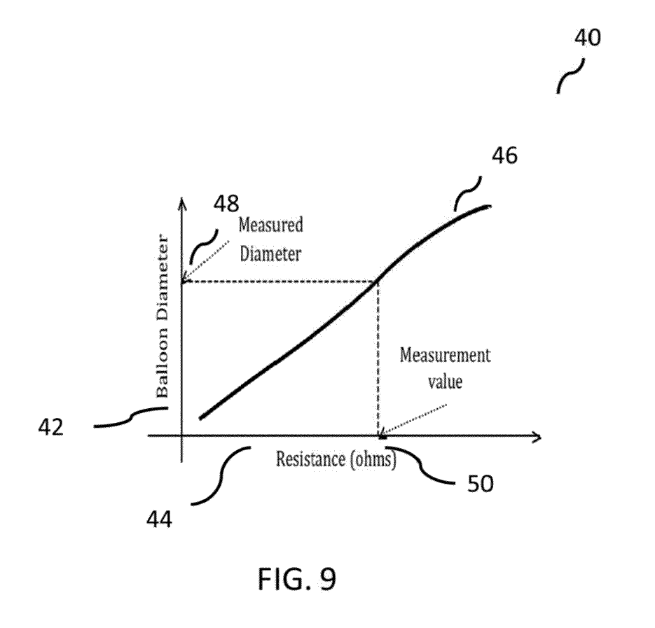

A graphical representation 40 is shown in FIG. 9 that shows a relationship between the measured resistance on the axis 44 with the diameter of the balloon on the axis 42. Through this relationship, the measured resistance value 50 (observed attribute value) can directly be mapped to the diameter 48 (dimension) of the balloon. It would be appreciated by those skilled in the an that the measurement is not affected by the nature of the surrounding wall of the blood vessel nor the exact pressure of the fluid inside the balloon, and hence is more accurate than the prior art methodologies. As mentioned herein above the aspects of the invention include obtaining radial expansion profile, as well as longitudinal expansion profile. The longitudinal expansion of the balloon is a useful measurement as it would prevent the bulging out of the balloon beyond the stent that usually causes damage to the neighboring wall of the blood vessel.

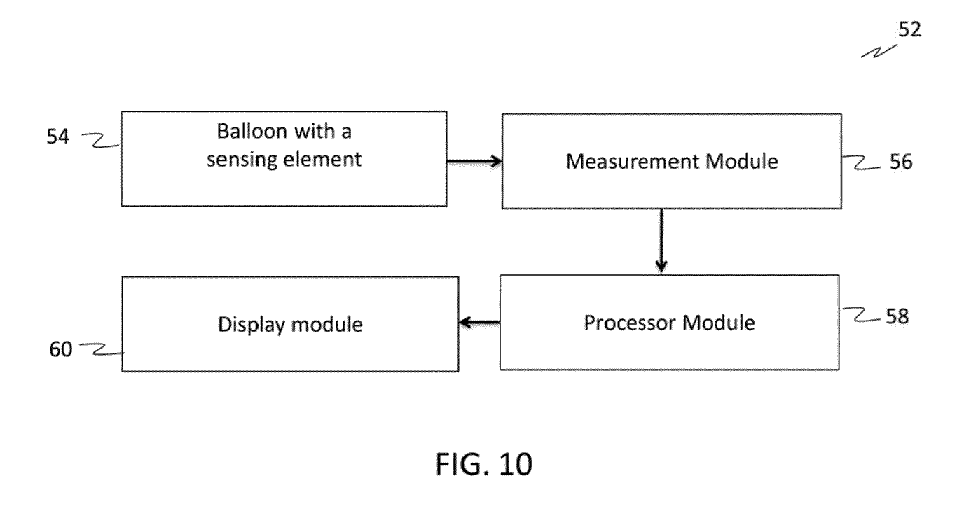

Another exemplary embodiment of the invention is a diagnostic kit 52 for measuring a balloon expansion profile in vivo as shown in FIG. 10. The diagnostic kit 52 includes a balloon 54 with a sensing element where the sensing element is characterized by at least one attribute that is representative of balloon dimension as explained in reference to FIG. 1. The diagnostic kit 52 further includes a measurement module 56 for measuring an observed attribute value for the attribute and a processor module 58 for processing, the observed attribute value to estimate the balloon expansion profile as one or more outputs. The processor module is further configured to compare the observed attribute value with a desired attribute value that is useful for further analysis and for guiding the medical procedure. In one exemplary embodiment, the processor module is further configured to estimate a direction in reference to a predefined location for the change in the balloon dimension. In an exemplary embodiment. the diagnostic kit 52 also includes a display module 60 to display the one or more outputs. The measurement module 56 described herein is further configured to measure a change in the balloon expansion profile that may happen during the medical procedure. The measurement module 56 is further configured to measure the observed attribute at a single location in one embodiment and at multiple locations in another exemplary embodiment.

As would be appreciated by those skilled in the art, the diagnostic device, method and the diagnostic kit as described herein increase the effectiveness of the medical procedures. This embodiments described herein can also be used in procedures other than cardiovascular such as peripheral arterial diseases. Further, the exemplary embodiments can be used in any application where a balloon like structure is used to expand a cavity using a fluid or gas pumped in to expand the balloon.

While only certain features of the invention have been illustrated and described herein, many modifications and changes will occur to those skilled in the art. It is, therefore, to be understood that the appended claims are intended to cover all such modifications and changes as fail within the true spirit of the invention.

What is claimed is:

1. A method for measuring a balloon expansion profile in vivo, the method comprising:
providing a balloon with at least one sensing element, wherein the at least one sensing element is characterized by at least one attribute that is representative of balloon dimension;
measuring the at least one attribute to obtain an observed attribute value; and
estimating the balloon dimension and the balloon expansion profile based on the observed attribute value.

2. The method of claim I wherein the at east one sensing element comprises at least two electrodes.

3. The method of 2 wherein the at least one attribute is impedance between the at least two electrodes.

4. The method of claim 2 wherein the at least one attribute is a voltage difference measured between the at least two electrodes.

5. The method of claim 2 wherein the at least one attribute is resonance frequency between the at least two electrodes.

6. The method of claim 1 wherein the at least one attribute is used to measure a change in the balloon dimension.

7. The method of claim 6 further comprising estimating a direction in reference to a predefined location for the change in the balloon dimension.

8. The method of claim 6 wherein the change is representative of an expansion of the balloon.

9. The method of claim 1 wherein the balloon expansion profile is a dimension along one axis of the balloon.

10. The method of claim 1 further comprising measuring the observed attribute at a single location.

11. The method of claim 1 further comprising measuring the observed attribute at a plurality of locations.

12. A diagnostic kit for measuring a balloon expansion profile in vivo, the diagnostic kit comprising:
a balloon with at least one sensing element, wherein the at least one sensing element is characterized by at least one attribute that is representative of balloon dimension
a measurement module for measuring an observed attribute value for the attribute and
a processor module for processing the observed attribute value to estimate the balloon expansion profile as one or more outputs.

13. The diagnostic kit of claim 12 further comprising a display module to display the one or more outputs.

14. The diagnostic kit of claim 12 wherein the processor module is further configured to compare the observed attribute value with a desired attribute value.

15. The diagnostic kit of claim 12 wherein the at least one sensing element comprises at least two electrodes.

16. The diagnostic kit of claim 15 wherein the at least one attribute is electrical resistance between the at least two electrodes.

17. The diagnostic kit of claim 15 wherein the at least one attribute is electrical capacitance between the at least two electrodes.

18. The diagnostic kit of claim 15 wherein the at least one attribute is resonance frequency between the at least two electrodes.

19. The diagnostic kit of claim of claim 12 wherein the at least one sensing element is mounted on the surface of the balloon.

20. The diagnostic kit of claim 12 wherein the at least one sensing element is present inside the balloon.

21. The diagnostic kit of claim 12 wherein the measurement module is further configured to measure a change in the balloon expansion profile.

22. The diagnostic kit of claim 21 wherein the processor module is further configured to estimate a direction in reference to a predefined location for the change in the balloon dimension.

23. The diagnostic kit of claim 12 wherein the measurement module is further configured to measure the observed attribute at a single location.

24. The diagnostic kit of claim 12 wherein the measurement module is further configured to measure the observed attribute at a plurality of locations.

25. The diagnostic kit of claim 12 wherein the at least one sensing element is an integral component of the balloon.

26. The diagnostic kit of claim 25 wherein the at least one sensing element is a piezoelectric material integrated in the balloon.

27. The diagnostic kit of claim 25 wherein the at least one sensing element is a capacitive element embedded in a wall of the balloon.

28. The diagnostic kit of claim 12 wherein the at least one sensing element is an elastic resistive element embedded along at least a portion of a circumference on a surface of the balloon.

29. A diagnostic device comprising a balloon having at least one sensing element for measuring at least one balloon expansion profile.

30. The diagnostic device of claim 29 wherein the at least one sensing element comprises at least two electrodes.

31. The diagnostic device of claim 29 wherein the at least one sensing element is an integral component of the balloon.

32. The diagnostic device of claim 31 wherein the at least one sensing element a piezoelectric material integrated in the balloon.

33. The diagnostic device of claim 31 wherein the at least one sensing element is a capacitive element embedded in a wall of the balloon.

34. The diagnostic device of claim 29 wherein the at least one sensing element is an elastic resistive element embedded along at least a portion of a circumference on a surface of the balloon.
